(19) United States
(12) Patent Application Publication
(10) Pub. No.: US 2022/0065699 A1
(43) Pub. Date: Mar. 3, 2022

(54) Cell Phone with Digital Temperature Scanner

(71) Applicant: Roger Pawson, San Diego, CA (US)

(72) Inventor: Roger Pawson, San Diego, CA (US)

(21) Appl. No.: 17/459,324

(22) Filed: Aug. 27, 2021

Related U.S. Application Data
(60) Provisional application No. 63/071,923, filed on Aug. 28, 2020.

(51) Int. Cl. G01J 5/02; A61B 5/01; G06K 9/00

(52) U.S. Cl. CPC G01J 5/0265; G01J 5/028; G06K 9/00288; A61B 5/01

(57) ABSTRACT

A handheld device with a temperature sensor incorporated into its body is taught. The temperature sensor is used to remotely take a person's temperature. The temperature sensor is powered by the handheld device's battery and is controlled by the touchscreen of the device. The device combines facial recognition through the camera lens with remote temperature sensing to provide a quick way to determine if a certain person presents a danger to himself/herself and others because of a high temperature.

### CROSS REFERENCE TO RELATED APPLICATIONS

This application claims priority back to U.S. Provisional No. 63/071,923, with a filing date of Aug. 28, 2020, the contents of which are incorporated by reference into this utility patent application.

### STATEMENT REGARDING FEDERALLY SPONSORED RESEARCH OR DEVELOPMENT

This invention was not federally sponsored.

### BACKGROUND OF THE INVENTION

### Field of the Invention

This invention relates to the general field of temperature taking devices, and more specifically to a cell phone or tablet into which a temperature taking device has been incorporated. The device allows a user to take temperatures with a “touchless method” by using a commonly-owned and commonly-carried device as opposed to have to bring along a specific thermometer or other temperature-taking device to perform the same function. The invention is particular adapted to intelligent passage systems such as those found in office complexes, clubs, sporting events, and other locations where rapid determination of whether a person has a dangerously high temperature is desirable.

### BRIEF DESCRIPTION OF INVENTION

The invention is a handheld device with a remote temperature sensor incorporated into its body. In a preferred embodiment, the senor is located in a socket on the front or back of the device. The technology is intended for use primarily in cellphones and watches, but is equally applicable to any device used in a location where remote temperature sensing is desirable. The temperature sensor can be attached to a wall or hand held, and can take a person's temperature by merely pointing the sensor at the forehead or other appropriate body part of the person whose temperature is being taken. The sensor is powered by the handheld device's battery and is controlled by the touchscreen of the device.

### Statement of the Problem

Taking a person's temperature has been an integral part of the medical practice for centuries. Since the recent Covid epidemic, however, the need for a sanitary method of taking temperatures has become even more important, with remote sensing of temperatures becoming even more desirable than pre-Covid. There exist a number of devices that can be used to take temperatures, but these devices either require a medical practitioner to touch the patient or are stand-alone units that perform a single function—the taking of temperatures remotely. Thus, there is a need for a device that incorporates the remote taking of temperature into its already-existing capacities, rather than a device that only takes temperature remotely.

The current invention provides just such a solution by having a remote temperature sensor built into the body of a handheld device such as a cellphone, tablet, or personal digital assistant. The sensor can be located in a socket on the surface of the device and controlled through the touchscreen, with power supplied by the device's battery. A guide temperature sensor can show the area to be sensed, and by simply pressing the “Temperature Sensor” icon on the screen, the person can activate the temperature sensor and direct it to the forehead or other appropriate area of the user. In a preferred embodiment, the sensor is located next to the device's flashlight, and controlled in a similar manner.

In one embodiment, the invention is a small, temperature taking device that can be removably mounted to a wall or other structure. In this embodiment, the device also has facial recognition capabilities that detect the identify of the person through facial recognition and then detects that specific person's temperature and stores it in a database.

### STATEMENT OF THE INVENTION

The invention is a device into which a temperature sensor has been incorporated into the device.

The temperature sensor is controlled by a touchscreen on the device into which it has been embedded and is powered by the device's battery.

A cellphone is an optimum device into which the Temperature sensor can be incorporated, but it can also be used in tablets, PDA's and other devices with a touchscreen which can be easily and removably mounted to a wall or other structure.

There has thus been outlined, rather broadly, the more important features of the invention in order that the detailed description thereof may be better understood, and in order that the present contribution to the art may be better appreciated. There are additional features of the invention that will be described hereinafter and which will form the subject matter of the claims appended hereto. The features listed herein and other features, aspects and advantages of the present invention will become better understood with reference to the following description and appended claims. The accompanying drawings, which are incorporated in and constitute part of this specification, illustrate embodiments of the invention and, together with the description, serve to explain the principles of the invention.

It should be understood the while the preferred embodiments of the invention are described in some detail herein, the present disclosure is made by way of example only and that variations and changes thereto are possible without departing from the subject matter coming within the scope of the following claims, and a reasonable equivalency thereof, which claims I regard as my invention.

### BRIEF DESCRIPTION OF THE FIGURES

One preferred form of the invention will now be described with reference to the accompanying drawings.

FIG. 1 is a front view of a cell phone that shows the location of various components of the invention.

FIG. 2 is a front, cutaway view of a cell phone illustrating how the various components of the invention are located.

FIG. 3 is a front view of the screen of a watch showing the location of the temperature sensor. This is explicitly not meant to be limiting as to the location on the sensor.

FIG. 4 is a cut-away view of the electrical components of the invention in the watch embodiment, showing how the invention appears opened up.

### DETAILED DESCRIPTION OF THE FIGURES

Many aspects of the invention can be better understood with references made to the drawings below. The components in the drawings are not necessarily drawn to scale. Instead, emphasis is placed upon clearly illustrating the components of the present invention. Moreover, like reference numerals designate corresponding parts through the several views in the drawings. Before explaining at least one embodiment of the invention, it is to be understood that the embodiments of the invention are not limited in their application to the details of construction and to the arrangement of the components set forth in the following description or illustrated in the drawings. The embodiments of the invention are capable of being practiced and carried out in various ways. In addition, the phraseology and terminology employed herein are for the purpose of description and should not be regarded as limiting.

It should be understood that while the preferred embodiments of the invention are described in some detail herein, the present disclosure is made by way of example only and that variations and changes thereto are possible without departing from the subject matter coming within the scope of the following claims, and a reasonable equivalency thereof, which claims I regard as my invention.

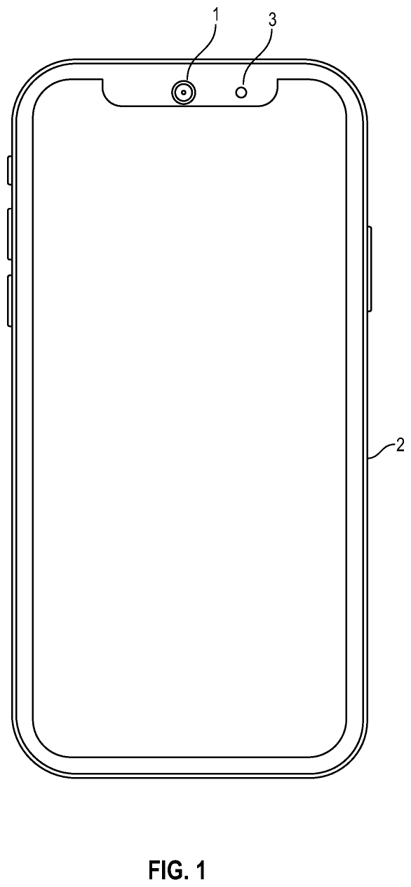

FIG. 1 is a front view of a cell phone that shows the location of various components of the invention. The device has a temperature detection lens 1, that is embedded into the body 2 of the handheld device. The handheld device 2 has, optionally, mounting means of the back such that the device can be removably mounted to a wall or other structure. The camera lens 3 is connected to facial recognition software that takes a picture of the face of the person whose temperature is going to be taken, then facial recognition software (either stored in the device or remotely accessed) identifies the person. The temperature detection lens 1 then takes the person's temperature, and the identity of the person, the person's temperature, and the time and date of the temperature taking are stored in a database. The database can be internal to the device or remotely stored. The device also has a high temperature triggering function, where is the person's temperature is above a pre-set “danger level”, the device notifies the person and the database of the temperature. A remote warning is also triggered such that a control unit that oversees the temperatures of persons whose temperatures are being taken is notified of the abnormally high temperature. The data can be transferred through USB/wired ports, BlueTooth or other wireless methods.

As a practical example, let's take an office building with numerous employees. When a shift begins, those working that shift line up in front of the remote temperature detection device. The temperature detection lens takes the person's temperature as the camera lens takes the person's pictures. The person's identity is quickly determined through facial recognition software and the identify of the person, his/her temperature, and the time/date of the temperature taking are transferred to a database. Should the person's temperature exceed a pre-set (and optionally, adjustable) number, the office security team is notified and the person is escorted out of the line for further evaluation.

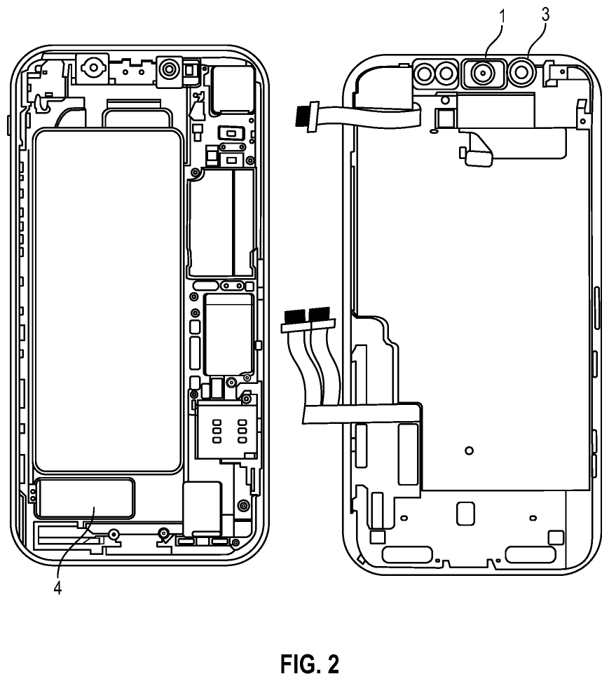

FIG. 2 is a front, cutaway view of a cell phone illustrating how the various components of the invention are located. The temperature detection lens 1 is located, in this embodiment, close the to camera lens 3. Both lenses are embedded into the body 2 of the handheld device. Power is supplied by a battery 4.

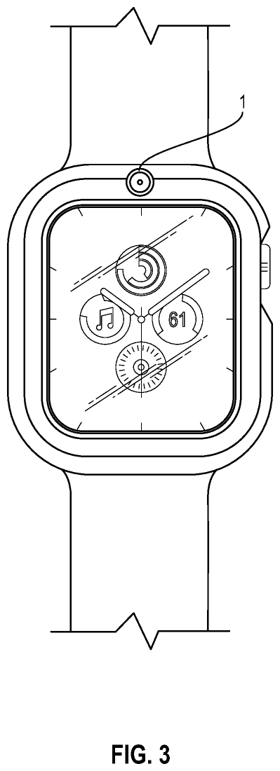

FIG. 3 is a front view of the screen of a watch showing the location of the temperature sensor. This is explicitly not mean to be limiting as to the location on the sensor. The temperature detection lens 1 is located, in this embodiment, at the top of the body 2 of the watch.

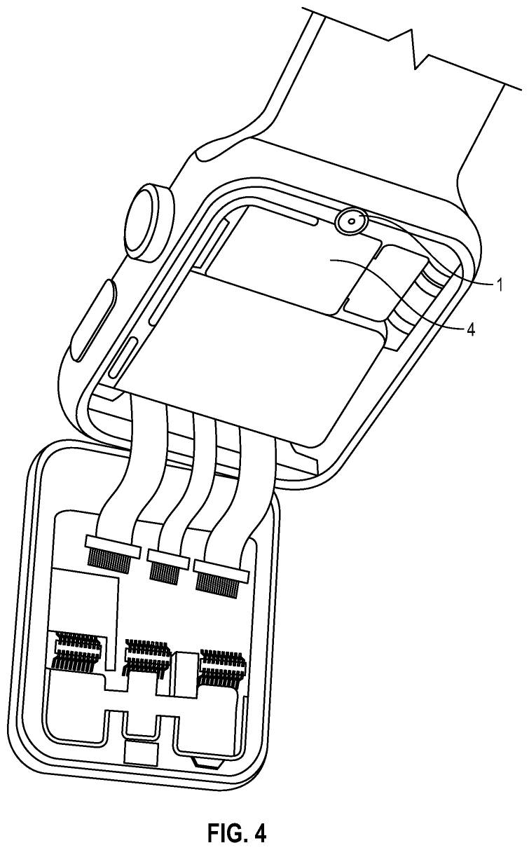

FIG. 4 is a cut-away view of the electrical components of the invention in the watch embodiment, showing how the invention appears opened up. Power is supplied by a battery 4.

There are a number of optional and variable elements to the invention. For example, each device may have internal storage, or can be wirelessly or directly wired to an external device that performs the facial recognition and stores the data obtained from the temperature detection lens and the camera lens. The device can be permanently mounted to a wall, with glue, sticky backing or screws, or removable either directly removable from the wall, or removable from a mount that is more permanently attached to the wall. It is also contemplated that the device can be mounted on a tripod or other elevation means in a turnstile location such that minimal changes need to be made the existing structure to locate the device at head level.

The system management is done primarily on the screen 4 of the body 2 of the device. A pop-up screen allows a system management user a number of options. The temperature records can be sorted by individual, by day, or by any other relevant factor, but that the overall health of a population or workers or attendees can be evaluated over time.

The process normally begins with a Registration Screen, by which a new person is registered and his/her picture is digitally associated with his/her data. As an added security feature, a worker may be assigned an individualized password such that he/she has to pass both an “personal identity” test and a “facial recognition” identity test. All of these parameters can be set and adjusted by the “department settings”, which are controlled by the security branch of a particular office complex or sporting venue. The time of check-in and check-out can also be correlated with the pay of an office complex worker.

All of the material in this patent document is subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

What I claim is:

1. A remote temperature taking device, consisting of: a temperature sensor, a housing with a camera lens, a source of power and a control chip, where, the housing removably attaches the temperature sensor to a wall, turnstile or other permanent structure, where the source of power powers the temperature sensor, where the control chip takes a first signal from a touchscreen icon and allows power from the source of power to turn on the temperature sensor, and where a picture taken by the camera lens is transmitted to a facial recognition software, and where the facial recognition software identifies a person in front of the camera lens, and where the temperature sensor takes a temperature reading of the person in front of the camera lens, and where the temperature reading of the person in front of the camera lens is transmitted to a database, and where a danger range of temperature is established, and where, when the temperature reading of the person in front of the camera lense is in the danger range, a security department is notified.

2. The device of claim 1, where there is a touchscreen sensor icon by which a user can activate the temperature sensor and the camera lens.

3. The device of claim 2, where the housing is a cell phone housing.

4. The device of claim 2, where the housing is a watch housing.
The device of claim 2, where the housing is a tablet housing.
